(19) United States
(12) Patent Application Publication
(10) Pub. No.: US 2004/0067896 A1
(43) Pub. Date: Apr. 8, 2004

(54) Novel pseudoerythromycin derivatives

(75) Inventors: Satoshi Omura, Tokyo (JP); Yuzuru Iwai, Tokyo (JP); Toshiaki Sunazuka, Tokyo (JP); Tohru Nagamitsu, Tokyo (JP)

Correspondence Address: YOUNG & THOMPSON, 745 SOUTH 23RD STREET 2ND FLOOR, ARLINGTON, VA, 22202

(73) Assignee: THE KITASATO INSTITUTE, TOKYO (JP)

(21) Appl. No.: 10/663,669

(22) Filed: Sep. 17, 2003

Related U.S. Application Data
(62) Division of application No. 10/088,965, filed on Jul. 25, 2002.
Application No. 10/088,965 is a 371 of international application No. PCT/JP00/05503, filed on Aug. 17, 2000.

(51) Int. Cl. C07H 17/08; A61K 31/7048

(52) U.S. Cl. 514/28; 536/7.1

(57) ABSTRACT

The present invention is to obtain novel anti-inflammatory agents having decreased antibacterial activity and increased anti-inflammatory action, and is psedoerythromycin derivatives represented by the following general formula [I],

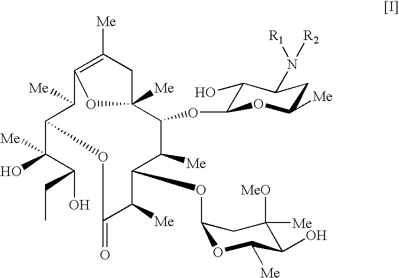

wherein R1 and R2 are same or different and each represents H, alkyl, alkynyl, acyl or sulfonyl, in which these groups may optionally have substituents, and Me indicates methyl.

### CROSS REFERENCE TO RELATED APPLICATION

This application is a Divisional of U.S. patent application Ser. No. 10/088,965 filed on Jul. 25, 2002. application Ser. No. 10/088,965 is the national phase of PCT International Application No. PCT/JP00/05503 filed on Aug. 17, 2000 under 35 U.S.C. § 371.

### BACKGROUND OF THE INVENTION

1. Field of the Invention

The present invention relates to novel pseudoerythromycin derivatives or salt thereof.

2. Description of Related Art

Erythromycin (hereinafter sometimes designates as EM) has been used for long time as 14-membered macrolide antibiotic for treatment of infectious disease caused by Gram-positive bacteria. During past ten and several years, erythromycin has known to improve long-term chronic inflammatory diseases such as diffuse panbronchiolitis and bronchial asthma, except for therapeutic action to bacterial infectious diseases. (Kudo, Shoji et al., Studies of clinical results on long term small administration of erythromycin for diffuse panbronchiolitis-Treatment results for 4 years, J. Japan. Thorac. Dis. Assoc., 25:632-642, 1987).

Erythromycin is an antibiotic and has antibacterial action which is not always required for treatment of inflammatory diseases. Consequently, resistant bacteria are generated in patients who are administered antibiotics, as a result, it causes deterioration for treatment of infectious disease in the other occasion.

As described above, Kudo, Shoji et al. demonstrated that diffuse panbronchiolitis could be improved by long term small administration of erythromycin (Kudo, Shoji et al., Studies of clinical results on long term small administration of erythromycin for diffuse panbronchiolitis-Treatment results for 4 years, J. Japan. Thorac. Dis. Assoc., 25:632-642, 1987).

### SUMARRY AND OBJECT OF THE INVENTION

Recently, actions other than antibiotic activity of erythromycin is noted, as a result, usefulness other than pulmonary region, for example not limited in diffuse panbronchiolitis but for chronic sinusitis and Crohn's disease are reported. The mechanism of action of erythromycin for chronic disease such as diffuse panbronchiolitis is thought to be the result of original antibacterial action. Research studies are now ongoing, and indicate the antiinflammatory action mediated by immune inflammatory cells in the penumbral chronic respiratory tract inflammation.

For example, the studies indicate the regulation for migration of neutrophils to infectious region by direct action, and the regulation for migration of neutrophils or for release of active oxygen from neutrophils by indirect action through mediators or cytokines. Further, erythromycin has an action to lymphocytes, macrophages and mast cells to regulate their proliferation or cytokine production, or to induce differentiation. (Kudo, Shoji Ed., Supervisors: Shimizu, Kihachiro and Omura Satoshi “Inflammation, Immunity and Macrolides Up to Date”, Iyaku Journal Inc., Osaka, 1996)

As explained above, 14-membered macrolides are thought to cure chronic respiratory diseases as a result of exhibiting immune regulation and antiinflammatory action.

We have aimed at the promoting action of erythromycin for differentiation-induction from monocyte to macrophage (N. Keicho, S. Kudoh, H. Yotsumoto, K. Akagawa, “Erythromycin promotes monocyte to macrophage differentiation”, J. Antibiotics, 47, 80-89, 1994), and tried to synthesize erythromycin derivatives for the purpose of creating the derivatives having disappeared antibacterial action and enhanced promoting action for differentiation-induction.

The present invention relates to a novel pseudoerythromycin derivative represented by the general formula [I],

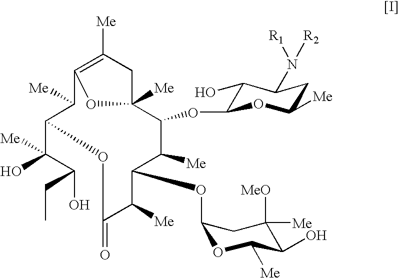

wherein R₁ and R₂ are same or different and each represents H, alkyl, alkynyl, acyl, or sulfonyl, in which these groups may optionally have substituents, and Me indicates methyl.

Further, the present invention relates to a novel pseudoerythromycin derivative represented by the general formula [II],

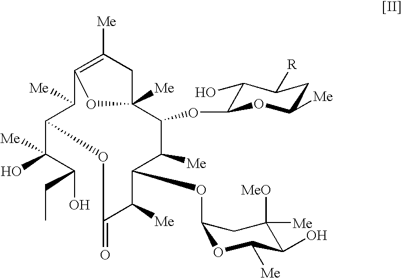

wherein R is heterocyclic containing N which may optionally have substituents, and Me indicates methyl.

The present invention further relates to a novel pseudo erythromycin derivative represented by the general formula [III],

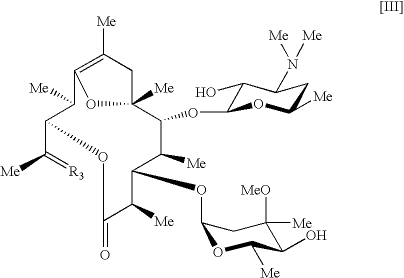

wherein R₃ is 0 or NOH, and Me indicates methyl.

The invention further relates to a novel pseudoerythromycin derivative represented by the general formula [IV],

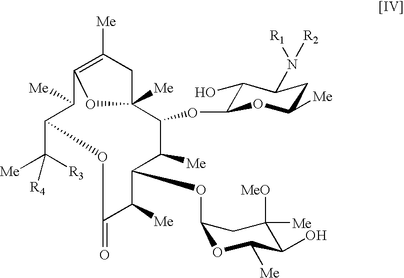

wherein R₁ and R₂ are same or different and each represents H or methyl, R₃ and R₄ represent H, hydroxyl or amino, and Me indicates methyl.

The present invention further relates to a novel pseudo erythromycin derivative represented by the general formula [V],

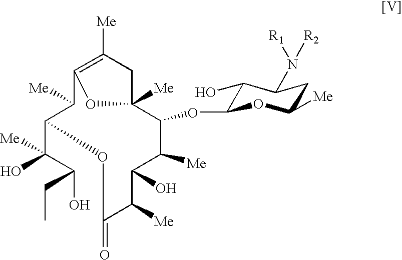

wherein R₁ and R₂ are same or different and each represents H or methyl, and Me indicates methyl.

Synthetic methods of various erythromycin derivatives are, for example, illustrated in the synthetic scheme as shown in FIG. 1. Namely, erythromycin A is treated with ice-cold acetic acid according to the references: (a) I. O. Kibwage, R. Busson, G. Janssen, J. Hoogmartens, H. Vanderhaeghe, Translactonization of Erythromycins, J. Org. Chem., 52, 990-996, 1987, (b) H. A. Kirst, J. A. Wind, J. W. Paschal, Synthesis of Ring-Constracted Derivatives of Erythromycin, J. Org. Chem., 52, 4359-4362, 1987, introducing to erythromycin A enol ether (EM 201), subsequently refluxing in methanol with heating in the presence of potassium carbonate to introduce pseudoerythromycin A enol ether (EM701) (known compound).

The product was treated with iodine and sodium acetate according to the reference (L. A. Friberg, U.S. Pat. No. 3,725,385) to obtain de-N-methyl-pseudoerythromycin A enol ether (EM703) (known compound). The compound was further treated with iodine and sodium methoxide to obtain bis(de-N-methyl)-pseudo erythromycin A enol ether (EM721) (novel compound). Alkylation, acylation and sulfonylation using EM703 and EM721 resulted to synthesize various derivatives through bis-de(3′-N-methyl)-3′-N-ethyl-8, 9-anhydro-pseudoerythromycin A 6, 9-hemiketal (EM722).

The synthetic scheme of compounds of the present invention is illustrated in FIG. 1. Namely, the compounds can be obtained by the synthetic route of: erythromycin A (EMA)→erythromycin A enol ether (EM201)→pseudoerythromycin A enol ether (EM701)→de-N-methyl-pseudoerythromycin A enol ether (EM703)→bis (de-N-methyl)-pseudoerythromycin A enol ether (EM721).

In order to confirm enhancing effect for differentiation-induction of the compounds of the present invention, the enhancing effect for differentiation-induction from human monocyte to macrophage was assayed. Method is performed as follows.

THP-1 cells were collected from cultured liquid by centrifugation, adjusted the concentration to 2×10⁵ cells/ml using medium (RPMI 1640) and distributed into the 48-well plate at 500 μl/well. PMA solution 10 μl and sample solution 5 μl were added in each well, stirred with mild shaking and incubated at 37° C. for 72-96 hours under 5% CO₂. Further MTT 0.5 mg/ml solution was added at 300 μl/well, and incubated at 37° C. for 3 hours under 5% CO₂. Supernatant solution was suctioned using the injection tube, added DMSO 500 μl using automatic continuous injector to dissolve formazan completely and transferred each 100 μl to the 96-well plate. The optical absorption was measured using the plate-reader.

Results of the enhancing effect for differentiation-induction from human monocyte to macrophage measured by the above assay method are shown in Table 1.

TABLE 1
Structure of EM703 analogous derivatives and activities in THP-1/Mφ system

| | |
|---|---|
| Others | Treated conc. (μM) |

| EM | R₁ | R₂ | 0.3 | 1 | 3 | 10 | 30 | ED₅₀ (μM)* |
|---|---|---|---|---|---|---|---|---|
| 703 | Me | H | + | + | + | + | / | 0.3 |
| 721 | H | H | NT | NT | − | + | / | 10 |
| 722 | Et | H | − | + | + | ++ | / | 1 |
| 723 | Et | Et | − | + | + | | / | 1 |
| 724 | Allyl | H | − | + | + | ++ | / | 1 |
| 725 | Allyl | Allyl | NT | − | ± | + | / | 3 |
| 726 | Ac | H | − | − | − | − | − | − |
| 727 | Ms | Me | − | + | + | + | + | 1 |
| 728 | CH₂C≡CH | H | − | + | + | + | + | 1 |
| 729 | CH₂C≡CH | CH₂C≡CH | − | ± | ± | ± | / | 1 |
| 730 | Pr | H | + | + | + | / | / | 0.3 |
| 731 | Pr | Pr | − | − | + | / | / | 3 |
| 732 | Bn | H | + | + | + | + | / | 0.3 |
| 733 | Bn | Bn | − | ± | ± | / | / | 1 |

| | | | | | | | |
|---|---|---|---|---|---|---|---|
| 734 | 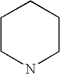 | − | ± | + | + | / | 1 |
| 735 | 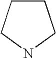 | − | ± | ++ | / | 1 | |

| | | | | | | | | |
|---|---|---|---|---|---|---|---|---|
| 736 | i-Pr | H | − | ± | + | ++ | / | 1 |
| 737 | Me | Me decladinose | NT | NT | − | + | / | 10 |
| 738 | C₆H₁₃ | H | − | ± | + | / | / | 1 |
| 739 | C₆H₁₃ | C₆H₁₃ | − | ± | + | + | / | 1 |
| 740 | C₂H₄F | Me | ± | ± | + | + | + | 0.3 |
| 742 | CH₂CN | Me | − | − | − | + | + | 10 |
| 743 | Me | Me Cl2oxime | NT | − | ± | − | / | − |
| 744 | C₃H₆OH | Me | NT | − | − | − | / | − |
| 745 | C₂H₄OAc | Me | − | − | ++ | ++ | ++ | 3 |
| 746 | Me | Me Cl2MeCHOH | − | ± | + | + | + | 1 |

| | | | | | | | |
|---|---|---|---|---|---|---|---|
| 747 | 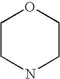 | NT | NT | − | ± | ++ | 10 |
| 748 | 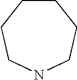 | − | ± | ++ | ++ | / | 1 |

| | | | | | | | | |
|---|---|---|---|---|---|---|---|---|
| 749 | (CH₂)₁₀Br | (CH₂)₁₀Br | NT | ± | + | + | /insolule | 1 |
| 750 | Me | Me Cl2MeCHNH₂ | NT | − | − | ± | / | 10 |
| 751 | H | Me Cl2MeCHOH | ± | ± | + | + | / | 0.3 |
| 754 | Me | H decladinose | NT | − | − | NT | + | 30 |
| EM | Me | Ml | NT | − | ± | + | + | 3 |
| CAM | Me | Ml | NT | NT | − | + | − | 10 |
| EM oxim | Me | Me C9oxime | NT | − | ± | ± | ++ | 3 |

In Table 1: Me:methyl; Pr:propyl; Et:ethyl; Ac:acetyl; and Ms:methanesulfonyl. *ED₅₀:Drug concentration (μM) required for 50% differentiation-induction of THP in Mφ.

In Table 1, indicated activity is represented in comparison with enhancing action for differentiation-induction of EM 100 μM, and symbols are: ++: enhanced 100% or more; +: enhanced 50-100%; ±: enhanced 25-50%; −: no activity; /: expressed cytotoxicity; and NT: not tested or under assessment.

As shown in Table 1, since the smaller the value of ED₅₀ (μM) (minimum drug concentration required for 50% differentiation-induction from THP-1 to Me), the stronger the differentiation-induction activity, it was found that the compounds of the present invention have enhancing action for differentiation-induction from THP-1 to Mφ.

Next, the suppressive effect of the compound of the present invention (EM703) against bleomycin-induced pulmonary fibrosis was examined (hereinafter sometimes designates bleomycin as BLM).

A sample suspended in 5% gum arabic was orally administered, 50 mg/kg/day for 17 days (from day-3 to day-13), and bleomycin, 100 mg/kg, was administered from tail vein in day-0. on day-28, animals were sacrificed under anesthesia and fibrosis of the lungs was compared with non-administered mice. Suppressive effects are shown in Table 2.

References:

Azuma A., Furuta T., Enomoto T., Hashimoto Y., Uematsu K., Nukariya N., Murata A., Kudoh S., Preventive effect of erythromycin on experimental bleomycin-induced acute lung injury in rats Thorax 53, 186-189, 1998

TABLE TWO
[Administration schedule]

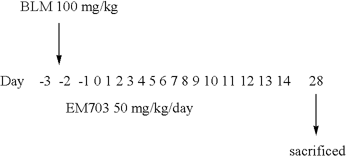

Results: Hydroxyproline levels in tissue

| | | Assay result | Weight conversion |
|---|---|---|---|
| Group | | (μmol/l) | (μmol/g) |
| Cont | | 440 | 4.0 |
| BLM | 1 | 785 | 7.1 |
| BLM | 2 | 733 | 6.4 |
| EM703 | 1 | 552 | 5.0 |
| EM703 | 2 | 489 | 4.6 |
| EM703 | 3 | 591 | 5.4 |
| BLM + EM703 | 1 | 583 | 5.2 |
| BLM + EM703 | 2 | 495 | 4.5 |
| BLM + EM703 | 3 | 437 | 4.4 |
| BLM + EM703 | 4 | 314 | 2.9 |
| BLM + EM703 | 5 | | |

Group:
Cont (control) group (n = 1)
BLM (bleomycin) group (n = 2)
EM (erythromycin) group (n = 4)
BLM (bleomycin) + EM (erythromycin) 703 group (n = 5)

As indicated above, hydroxyproline is an index of lung fibrosis and higher value indicates hyperfibrosis. Hydroxyproline level, an index for lung injury, in BLM administered group was reduced in a group of BLM+EM703.

Next, the suppressive effect of the compound EM703 against pneumonia caused by influenza viral infection was examined.

Sample was dissolved in physiological saline containing 1% DMSO and amount corresponding to oral dosage of the small administration for long-term therapy was administered from day-1 to day-6 of the infection to mice influenza pneumonia model (0.3 mg and 0.03 mg/mice), once a day, intraperitoneally. Results were compared with control group which was given only solvent.

Reference:

Sato K., Suga M., Akaike T. et al., Therapeutic effect of erythromycin on influenza virus-induced lung injury in mice. Am. J. Respir Crit. Care Med. 157, 853-859, 1998.

Results are shown in FIG. 2 and FIG. 3. In this system, mice developed pneumonia and almost died about 20 days after infection. Contrary to that, as shown in FIG. 2, administration of EM703, 0.3 mg/mice, cured pneumonia and 40% of mice were survived. Further, as shown in FIG. 3, mice without administration of drugs (control) indicated significant decrease of body weight due to pneumonia, but administration of EM703 indicated to increase body weight from day-10. This indicates suppressive effect against pneumonia and result to cure pneumonia.

As described above, the compound of the present invention shows suppressive effect against influenza virus-induced pneumonia.

### BRIEF DESCRIPTION OF THE FIGURES

FIG. 1 shows an example of the synthetic scheme of the compound of the present invention.

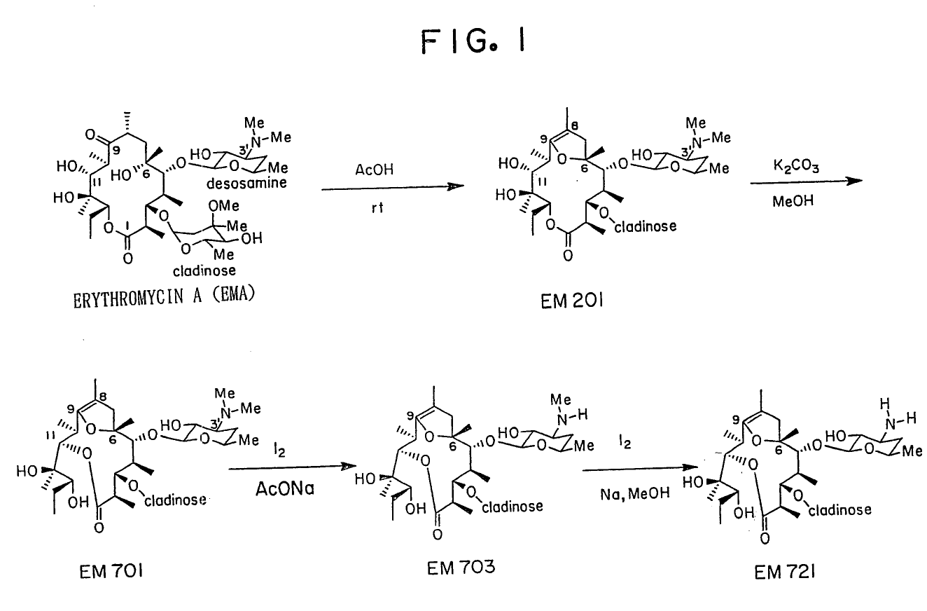

FIG. 2 is a graph of the suppressive effect against pneumonia showing relationship between numbers of day after infection due to influenza virus infection and survival rates of the compound of the present invention.

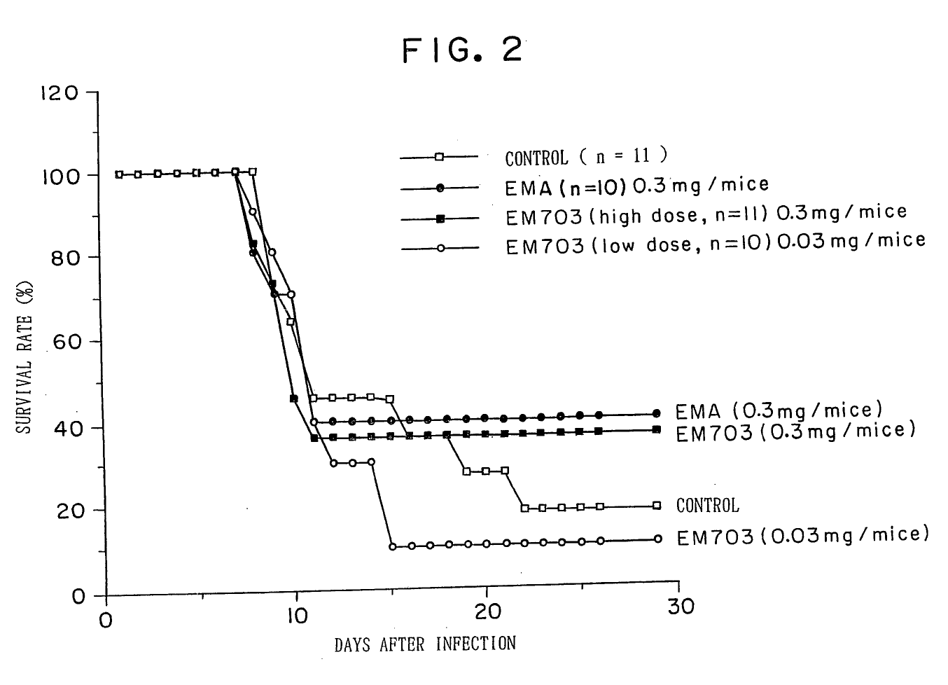

FIG. 3 is a graph showing suppressive effect of the compound of the present invention on bleomycin-induced pulmonary fibrosis.

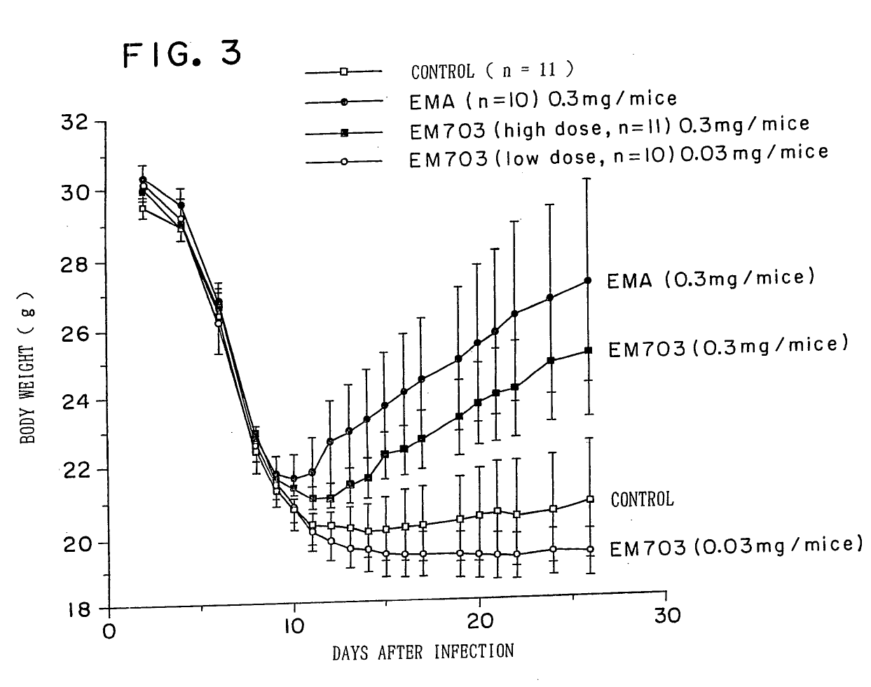

### DETAILED DESCRIPTION OF PREFERRED EMBODIMENTS

The present invention is explained by illustrating referential examples and examples, but is not limited within these examples.

### REFERENTIAL EXAMPLE 1

Synthesis of EM701

Glacial acetic acid solution of erythromycin A (12.4 g, 16.9 mmol) was stirred at room temperature for 2 hours, added slowly aqueous sodium hydrogen carbonate and neutralized. The reaction mixture was extracted with chloroform, dehydrated the organic layer with sodium sulfate, filtered off the sodium sulfate and removed the solvent by distillation to obtain crude substance. The crude substance was purified with silica gel chromatography (chloroform:methanol:aqueous ammonia=10:0.5:0.01→10:1:0.05) to obtain EM201 (7.7 g, 63%). Subsequently, potassium carbonate (1.4 g, 10.6 mmol) was added to the methanol solution (100 ml) of EM 201 (7.6 g, 10.6 mmol) and refluxed for 2 hours. After distilled off the solvent, the residue was dissolved in aqueous sodium hydrogen carbonate and extracted with chloroform. The mixture was dehydrated with sodium sulfate, filtered and removed the sodium sulfate, then the obtained crude substance was purified by silica gel chromatography (chloroform:methanol:aqueous ammonia=10:0.5:0.01→10:1:0.05) to obtain EM701 (5.9 g, 78%, white powder).

### EXAMPLE 1

Synthesis of De(3′-N-Methyl)-8, 9-Anhydro-Pseudoerythromycin A 6, 9-Hemiketal (EM703)

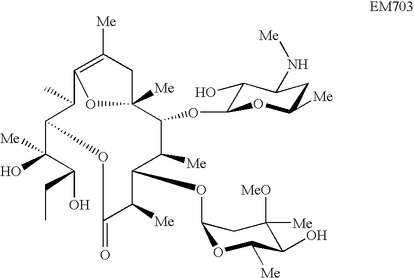

Sodium acetate (3.9 g, 48.5 mmol) and iodine (2.5 g, 9.7 mmol) were added in this order to methanol (52.0 mL)-water (13.0 mL) solution of EM701 (6.9 g, 9.7 mmol) at room temperature, and stirred at 50° C. for 3 hours. During the stirring, 1N aqueous solution of sodium hydroxide was added to maintain at pH 8-9 continuously. After confirming the completion of the reaction by TLC, the reaction mixture was diluted with aqueous ammonia (7.5 mL)-water (200 mL), and extracted with dichloromethane. After dehydrating the organic layer with sodium sulfate, the sodium sulfate was removed by filtration and distilled off the solvent to obtain crude substance. The crude substance was purified by silica gel column chromatography (chloroform:methanol:aqueous ammonia=10:0.5:0.01→10:1:0.05) to obtain EM703 (4.8 g, Yield: 70%, white powder). EM703: m. p.: 177-180° C.

Example 1 is a known compound. This is shown at line 703 in Table 1.

### EXAMPLE 2

Synthesis of Bis-De(3′-N-Methyl)-8, 9-Anhydro-Pseudo Erythromycin A 6, 9-Hemiketal (EM721)

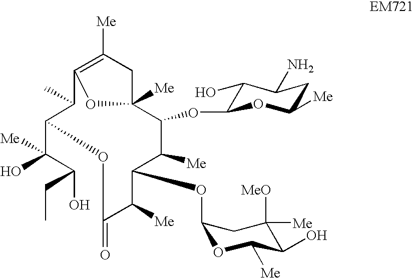

Sodium (4.5 g, 1.67 mmol) was added in methanol (15 mL) to prepare methanol solution of sodium methoxide, and EM703 (195.4 mg, 0.279 mmol) and iodine (353.6 mg, 1.393 mmol) were added in this order at 0° C. and stirred for 3 hours. After confirming completion of the reaction by TLC, sodium thiosulfate (0.8 g), aqueous ammonia (0.5 mL) and water (80 mL) were added and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM721 (166.3 mg, Yield: 87%, white powder).

| | |
|---|---|
| EM721: | m. p.: 134-136° C. |
| IR (KBr) ν: | 3467.4, 2973.7, 2935.1, 2879.2, 1700.9, |
| | 1637.3, 1457.9, 1380.8, 1265.1, 1166.7, |
| | 1126.2, 1079.9, 1037.5, 1016.3 cm⁻¹. |
| HRMS (FAB) m/z: | C₃₅H₆₁NO₁₂Na [M + Na]⁺ |

| | |
|---|---|
| Calculated | 710.4091, |
| Found | 710.4060. |

### EXAMPLE 3

Synthesis of Bis-De(3′-N-Methyl)-3′-N-Ethyl-8, 9-Anhydro-Pseudoerythromycin A 6, 9-Hemiketal (EM722)

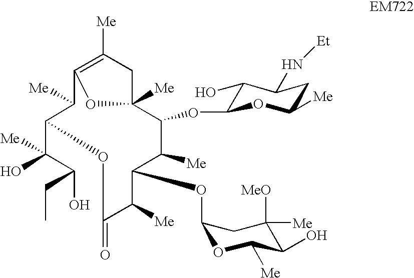

N,N-Diisopropylethylamine (26.6 μL, 0.153 mmol) and ethyl iodide (12.2 μL, 0.153 mmol) were added to dimethylformamide (1.0 mL) solution of EM721 (21.0 mg, 0.0305 mmol) and stirred at room temperature for 4 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM722 (7.0 mg, Yield: 32%, white powder).

| | |
|---|---|
| EM722: | m. p.: 124-126° C. |
| IR (KBr) ν: | 3471.6, 2933.2, 1704.8, 1457.9, 1378.9, |
| | 1263.1, 1166.7, 1128.2, 1074,2, 1037.5, |
| | 1018.2 cm⁻¹. |
| HRMS (FAB) m/z: | C₃₇H₆₅NO₁₂Na [M + Na]⁺ |

| | |
|---|---|
| Calculated | 738.4404 |
| Found | 738.4393. |

### EXAMPLE 4

Synthesis of Bis-De(3′-N-Methyl)-3′, 3′-N, N-Diethyl-8, 9-Anhydro-Pseudoerythromycin A 6, 9-Hemiketal (EM723)

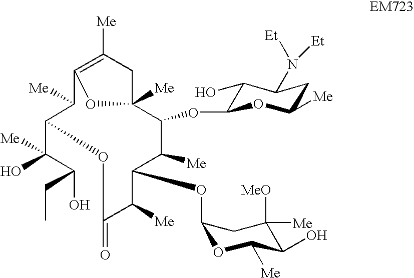

N,N-Diisopropylethylamine (26.6 μL, 0.153 mmol) and ethyl iodide (12.2 μL, 0.153 mmol) were added to dimethylformamide (1.0 mL) solution of EM721 (21.0 mg, 0.0305 mmol) and stirred at room temperature for 4 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM723 (10.3 mg, Yield: 45%, white powder).

| | |
|---|---|
| EM723: | m. p.: 165-168° C. |
| IR (KBr) ν: | 3473.7, 2935.1, 1699.0, 1382.7, 1317.1, |
| | 1267.0, 1166.7, 1126.2, 1108.9, 1078.0, |
| | 1016.3 cm⁻¹. |
| HRMS (FAB) m/z: | C₃₉H₆₉NO₁₂Na [M + Na]⁺ |

| | |
|---|---|
| Calculated | 766.4717 |
| Found | 766.4710. |

### EXAMPLE 5

Synthesis of Bis-De(3′-N-Methyl)-3′-N-Allyl-8, 9-Anhydro-Pseudoerythromycin A 6, 9-Hemiketal (EM724)

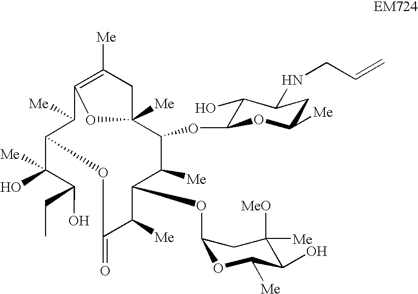

Allyl bromide (148.3 μL, 1.714 mmol) was added to dichloromethane (5.7mL) solution of EM721 (117.8 mg, 0.171 mmol) and N,N-Diisopropylethylamine (298.6 μL, 1.714 mmol) at 0° C. and stirred at room temperature for 2 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM724 (21.9 mg, Yield: 30%, white powder) was obtained.

| | |
|---|---|
| EM724: | m. p.: 106-109° C. |
| IR (KBr) ν: | 3448.8, 2971.8, 2933.2, 1718.3, 1637.3, |
| | 1380.8, 1265.1, 1166.7, 1126.2, 1078.0, |
| | 1037.5, 1016.3 cm⁻¹. |
| HRMS (FAB) m/z: | C₃₈H₆₅NO₁₂Na [M + N]⁺ |

| | |
|---|---|
| Calculated | 750.4404, |
| Found | 750.4420. |

### EXAMPLE 6

Synthesis of Bis-De(3′-N-Methyl)-3′, 3′-N, N-Diallyl-8, 9-Anhydro-Pseudoerythromycin A 6, 9-Hemiketal (EM725)

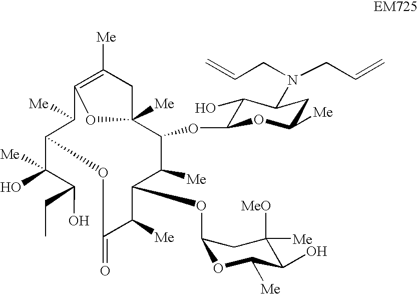

Allyl bromide (148.3 μL, 1.714 mmol) was added to dichloromethane (5.7 mL) solution of EM721 (117.8 mg, 0.171 mmol) and N,N-Diisopropylethylamine (298.6 μL, 1.714 mmol) at 0° C., stirred at room temperature for 2 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM725 (64.3 mg, Yield: 59%, white powder).

| | |
|---|---|
| EM725: | m. p.: 140-142° C. |
| IR (KBr) ν: | 3471.7, 2971.8, 2927.4, 1700.9, 1637.3, |
| | 1380.8, 1317.1, 1265.1, 1166.7, 1124.3, |
| | 1114.7, 1049.1, 1016.3 cm⁻¹. |
| HRMS (FAB) m/z: | C₄₁H₆₉NO₁₂Na [M + Na]⁺ |

| | |
|---|---|
| Calculated | 790.4717 |
| Found | 790.4716. |

### EXAMPLE 7

Synthesis of Bis-De(3′-N-Methyl)-3′-N-Acetyl-8, 9-Anhydro-Pseudoerythromycin A 6, 9-Hemiketal (EM726)

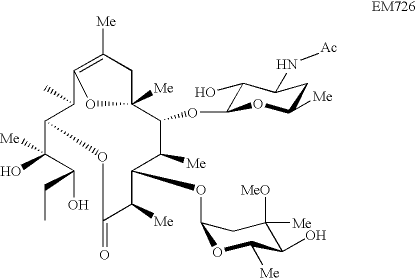

Acetic anhydride (8.4 μL, 0.0759 mmol) was added to dichloromethane (1.6 mL) solution of EM721 (34.8 mg, 0.0506 mmol) at 0° C., stirred for 10 minutes and further stirred at room temperature for 30 minutes. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol=100:1→20:1) to obtain EM726 (33.4 mg, Yield: 91%, white powder).

| | |
|---|---|
| EM726: | m. p.: 137-139° C. |
| IR (KBr) ν: | 3417.2, 2973.7, 2935.1, 1699.0, 1454.1, |
| | 1376.9, 1317.1, 1268.9, 1166,7, 1124.3, |
| | 1076.1, 1033.7, 1018.2, 1000.9 cm⁻¹. |
| HRMS (FAB)m/z: | C₃₇H₆₃NO₁₃Na [M + Na]⁺ |

| | |
|---|---|
| Calculated | 752.4197 |
| Found | 752.4202. |

### EXAMPLE 8

Synthesis of De(3′-N-Methyl)-3′-N-Sulfonyl-8, 9-Anhydro-Pseudoerythromycin A 6, 9-Hemiketal (EM727)

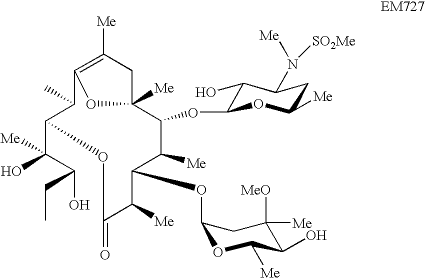

Methane sulfonyl chloride (9.3 μL, 0.249 mmol) was added to dichloromethane (4.2 ml) solution of EM703 (87.6 mg, 0.125 mmol) at 0° C. and stirred for 3 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol=100:1→20:1) to obtain EM727 (37.2 mg, Yield: 91%, white powder).

| | |
|---|---|
| EM727: | m. p.: 225-228° C. |
| IR (KBr) ν: | 3497.6, 2973.7, 2935.1, 1704.8, 1463.7, |
| | 1380.8, 1326.8, 1319.1, 1265,1, 1166.7, |
| | 1141.7, 1074.2, 1041.4, 1016.3 cm⁻¹. |
| HRMS (FAB)m/z: | C₃₇H₆₅NO₁₄SNa [M + Na]⁺ |

| | |
|---|---|
| Calculated | 802.4023 |
| Found | 802.3995. |

### EXAMPLE 9

Synthesis of Bis-De(3′-N-Methyl)-3′-N-Propargyl-8, 9-Anhydro-Pseudoerythromycin A 6, 9-Hemiketal (EM728)

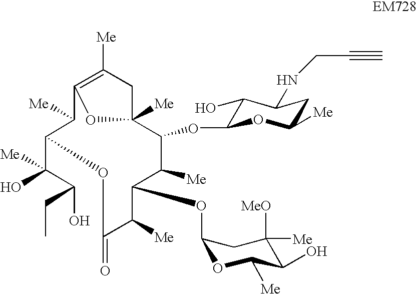

3-Bromopropine (137.8 μL, 1.546 mmol) was added to dichloromethane (5.2 mL) solution of EM721 (106.3 mg, 0.155 mmol) and N,N-Diisopropylethylamine (269.3 μL, 1.546 mmol), and stirred at room temperature for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM728 (41.3 mg, Yield: 37%, white powder).

| | |
|---|---|
| EM728: | m. p.: 113-115° C. |
| IR (KBr) ν: | 3413.0, 2973.7, 2935.1, 1706.8, 1457.9, |
| | 1382.7, 1263.1, 1166.7, 1126,2, 1078.0, |
| | 1039.4, 1016.5 cm⁻¹. |
| HRMS (FAB)m/z: | C₃₈H₆₃NO₁₂Na [M + Na]⁺ |

| | |
|---|---|
| Calculated | 748.4248 |
| Found | 748.4260. |

### EXAMPLE 10

Synthesis of Bis-De(3′-N-Methyl)-3′, 3′-N, N-Di-Propargyl-8, 9-Anhydro-Pseudoerythromycin A 6, 9-Hemiketal (EM729)

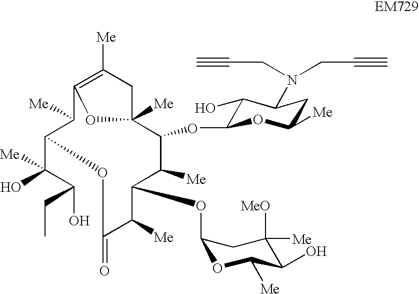

3-Bromopropine (137.8 μL, 1.546 mmol) was added to dichloromethane (5.2mL) solution of EM721 (106.3 mg, 0.155 mmol) and N,N-Diisopropylethylamine (269.3 μL, 1.546 mmol) and stirred at room temperature for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05) to obtain EM729 (27.9 mg, Yield: 24%, white powder).

| EM729: | m. p.: 123-125° C. | |
|---|---|---|
| IR (KBr) ν: | 3415.0, 3309.2, 2971.8, 2933.2, 2877.3, | |
| | 1706.7, 1457.9, 1375.0, 1263.1, 1166.7, | |
| | 1116.6, 1072.2, 1049.1, 1035.6, 1016.3 cm⁻¹. | |
| HRMS (FAB)m/z: | C₄₁H₆₅NO₁₂Na [M + Na]⁺ | |
| | Calculated | 786.4404 |
| | Found | 786.4404. |

### EXAMPLE 11

Synthesis of Bis-De(3′-N-Methyl)-3′-N-Propyl-8, 9-Anhydro-Pseudoerythromycin A 6, 9-Hemiketal (EM730)

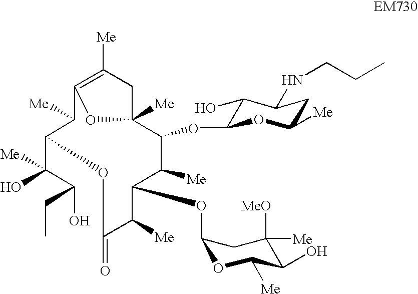

N,N-Diisopropylethylamine (59.6 μL, 0.342 mmol) and 1-iodopropane (33.3 μL, 0.342 mmol) were added in this order to acetinitrile (2.3 mL) solution of EM721 (23.5 mg, 0.0342 mmol) and refluxed at 80° C. for 20 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM730 (5.7 mg, Yield: 23%, white powder).

| | |
|---|---|
| EM730: | m. p.: 109-111° C. |
| IR (KBr) ν: | 3435.0, 2971.8, 2935.1, 2879.2, 1706.7, |
| | 1459.8, 1380.8, 1263.1, 1166.7, 1126.2, |
| | 1078.0, 1035.6, 1016.3 cm⁻¹. |
| HRMS (FAB)m/z: | C₃₈H₆₇NO₁₂Na [M + Na]⁺ |

| | |
|---|---|
| Calculated | 752.4560 |
| Found | 752.4564. |

### EXAMPLE 12

Synthesis of Bis-De(3′-N-Methyl)-3′, 3′-N, N-Di-Propyl-8, 9-Anhydro-Pseudoerythromycin A 6, 9-Hemiketal (EM731)

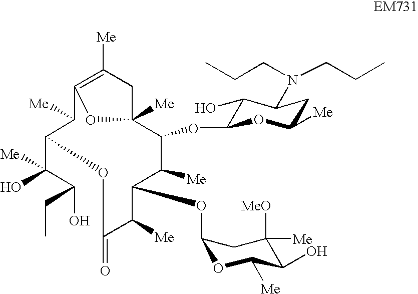

N,N-Diisopropylethylamine (59.6 μL, 0.342 mmol) and 1-iodopropane (33.3 μL, 0.342 mmol) were added in this order to acetinitrile (2.3 mL) solution of EM721 (23.5 mg, 0.0342 mmol) and refluxed at 80° C. for 20 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM731 (12.0 mg, Yield: 40%, white powder).

| EM731: | m. p.: 148-151° C. | |
|---|---|---|
| IR (KBr) ν: | 3435.0, 2964.1, 2933.2, 2873.4, 1706.7, | |
| | 1457.9, 1376.9, 1319.1, 1263.1, 1166.7, | |
| | 1110.8, 1081.9, 1049.1, 1035.6, 1016.3 cm⁻¹. | |
| HRMS (FAB)m/z: | C₄₁H₇₃NO₁₂Na [M + Na]⁺ | |
| | Calculated | 794.5030 |
| | Found | 794.5005. |

### EXAMPLE 13

Synthesis of Bis-De(3′-N-Methyl)-3′-N-Benzyl-8, 9-Anhydro-Pseudoerythromycin A 6, 9-Hemiketal (EM732)

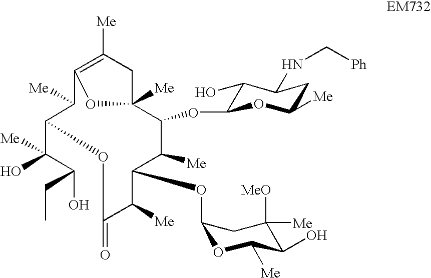

Benzyl chloride (297.3 μL, 2.584 mmol) was added to dichloromethane (4.3 mL) solution of EM721 (88.8 mg, 0.129 mmol) and N,N-diisopropylethylamine (450.1 μL, 2.584 mmol) at room temperature and stirred for 96 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM732 (49.9 mg, Yield: 50%, white powder).

| | |
|---|---|
| EM732: | m. p.: 126-128° C. |
| IR (KBr) ν: | 3410.0, 2971.8, 2935.1, 1706.7, 1456.0, |
| | 1378.9, 1263.1, 1166.7, 1124.3, 1078.0, |
| | 1049.1, 1039.4, 1016.3, 983.5, 937.2, |
| | 808.0, 752.1 cm⁻¹. |
| HRMS (FAB)m/z: | C₄₂H₆₇NO₁₂Na [M + Na]⁺ |

| | |
|---|---|
| Calculated | 800.4560 |
| Found | 800.4565. |

### EXAMPLE 14

Synthesis of Bis-De(3′-N-Methyl)-3′, 3′-N,N-Di-Benzyl-8, 9-Anhydro-Pseudoerythromycin A 6, 9-Hemiketal (EM733)

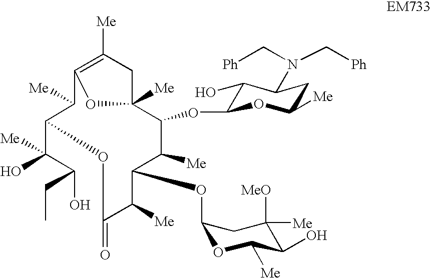

N,N-Diisopropylethylamine (135.9 μL, 0.780 mmol) and benzyl chloride (89.7 μL, 0.780 mmol) were added in this order to acetinitrile (1.3 mL) solution of EM721 (26.8 mg, 0.0390 mmol) and refluxed at 80° C. for 60 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM733 (19.6 mg, Yield: 58%, white powder).

| EM733: | m. p.: 149-152° C. | |
|---|---|---|
| IR (KBr) ν: | 3420.6, 2969.8, 2935.1, 1700.9, 1454.1, | |
| | 1375.0, 1324.9, 1263.1, 1166.7, 1116.6, | |
| | 1076.1, 1049.1, 1016.3, 752.1, 700.0 cm⁻¹. | |
| HRMS (FAB)m/z: | C₄₉H₇₃NO₁₂Na [M + Na]⁺ | |
| | Calculated | 890.5030 |
| | Found | 890.5032 |

### EXAMPLE 15

Synthesis of De(3′-Dimethylamino)-3′-Piperidino-8, 9-Anhydro-Pseudoerythromycin A 6, 9-Hemiketal (EM734)

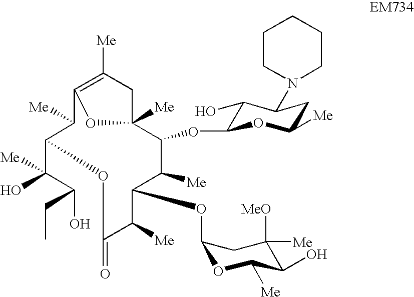

N,N-Diisopropylethylamine (42.5 μL, 0.244 mmol) and 1,5-dibromopentane (33.2 μL, 0.244 mmol) were added in this order to acetinitrile (4.9 mL) solution of EM721 (16.8 mg, 0.0244 mmol) and refluxed at 80° C. for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM734 (13.3 mg, Yield: 72%, white powder).

| | |
|---|---|
| EM734: | m. p.: 128-130° C. |
| IR (KBr) ν: | 3420.0, 2971.8, 2935.1, 2858.0, 1710.6, |
| | 1454.1, 1380.8, 1319.1, 1263.1, 1164.8, |
| | 1110.8, 1074.2, 1047.2, 1016.3 cm⁻¹. |
| HRMS (FAB)m/z: | C₄₀H₇₀NO₁₂ [M + Na]⁺ |

| | |
|---|---|
| Calculated | 756.4897 |
| Found | 756.4901 |

### EXAMPLE 16

Synthesis of De(3′-Dimethylamino)-3′-Pyrrolidino-8, 9-Anhydro-Pseudoerythromycin A 6, 9-hemiketal (EM735)

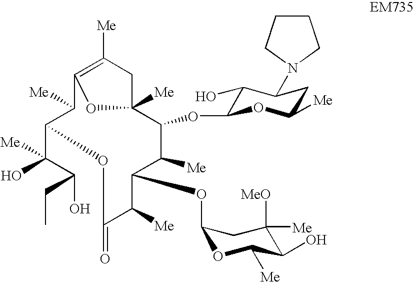

N,N-diisopropylethylamine (40.2 μL, 0.231 mmol) and 1,4-dibromobutane (27.6 μL, 0.231 mmol) were added in this order toacetinitrile (4.6 mL) solution of EM721 (15.9 mg, 0.0231 mmol) and refluxed at 80° C. for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:1:0.1) to obtain EM735 (11.9 mg, Yield: 70%, white powder).

| | |
|---|---|
| EM735: | m. p.: 127-129° C. |
| IR (KBr) ν: | 3420.0, 2971.8, 2937.1, 1702.8, 1457.9, |
| | 1382.7, 1265.1, 1166.7, 1124.3, 10761.1, |
| | 1049.1, 1016.3 cm⁻¹. |
| HRMS (FAB)m/z: | C₃₉H₆₈NO₁₂ [M + Na]⁺ |

| | |
|---|---|
| Calculated | 742.4741 |
| Found | 742.4743 |

### EXAMPLE 17

Synthesis of Bis-De(3′-N-Methyl)-3′-N-(2-Propyl)-8, 9-Anhydro-Pseudoerythromycin A 6, 9-Hemiketal (EM736)

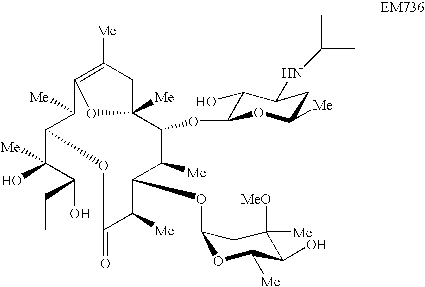

N,N-Diisopropylethylamine (459.2 μL, 2.636 mmol) and 2-bromopropane (247.5 μL, 2.636 mmol) were added in this order to acetinitrile (4.4 mL) solution of EM721 (90.6 mg, 0.132 mmol) and stirred at 80° C. for 72 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:1:0.1) to obtain EM736 (25.3 mg, Yield: 26%, white powder). The raw material EM721 was recovered 47.1 mg (Yield: 52%).

| | |
|---|---|
| EM736: | m. p.: 102-104° C. |
| IR (KBr) ν: | 3420.0, 2971.8, 2933.2, 2877.3, 1718.3, |
| | 1459.8, 1380.8, 1263.1, 1166.7, 1126.2, |
| | 1078.0, 1049.1, 1016.3 cm⁻¹. |
| HRMS (FAB)m/z: | C₃₈H₆₇NO₁₂Na [M + Na]⁺ |

| | |
|---|---|
| Calculated | 752.4560 |
| Found | 752.4576. |

Example 17 is a known compound. This is shown at line 736 in Table 1.

### EXAMPLE 18

Synthesis of De(3-O-Cladinosyl)-8, 9-Anhydro-Pseudo Erythromycin A 6, 9-Hemiketal (EM737)

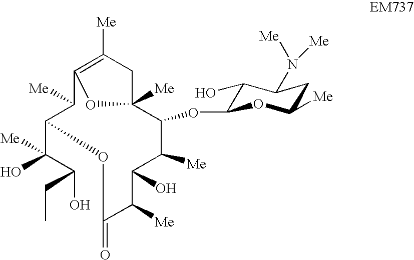

p-toluenesulfonic acid monohydrate (80.3 μL, 0.422 mmol) was added to dimethylformamide (5.6 mL) solution of EM701 (201.6 mg, 0.282 mmol) and stirred at 50° C. for 8 hours. After confirming completion of the reaction by TLC, the reaction mixture was diluted with water, adjusted to pH 8.0 by adding saturated aqueous sodium hydrogen carbonate and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM737 (84.7 mg, Yield: 54%, white powder)

| | |
|---|---|
| EM737: | m. p.: 109-111° C. |
| IR (KBr) ν: | 3486.7, 2973.7, 2937.1, 2877.3, 1708.6, |
| | 1631.5, 1457.9, 1382.7, 1265.1, 1164.8, |
| | 1110.8, 1076.1, 1039.4 cm⁻¹. |
| HRMS (FAB)m/z: | C₂₉H₅₂NO₉ [M + Na]⁺ |

| | |
|---|---|
| Calculated | 558.3641 |
| Found | 558.3616 |

### EXAMPLE 19

Synthesis of Bis-De(3′-N-Methyl)-3′-N-Hexyl-8, 9-Anhydro-Pseudoerythromycin A 6, 9-Hemiketal (EM738)

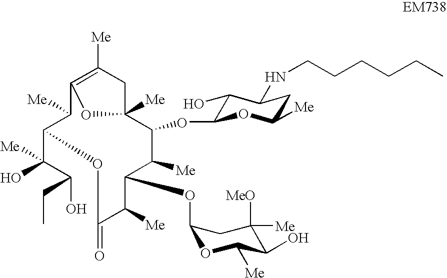

N,N-Diisopropylethylamine (408.5 μL, 2.345 mmol) and 1-bromohexane (328.7 μL, 2.345 mmol) were added in this order to acetinitrile (3.9 mL) solution of EM721 (80.6 mg, 0.117 mmol) and stirred at 60 ° C. for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM738 (33.7 mg, Yield: 45%, white powder). The raw material EM721 was recovered 24.6 mg (Yield: 31%).

| EM738: | m. p.: 115-118° C. | |
|---|---|---|
| IR (KBr) ν: | 3430.3, 2969.8, 2933.2, 2858.0, 1712.5, | |
| | 1459.8, 1378.9, 1317.1, 1263.1, 1166.7, | |
| | 1126.2, 1078.0, 1047.2, 1039.4, 1016.3 cm⁻¹. | |
| HRMS (FAB)m/z: | C₄₁H₇₄NO₁₂ [M + Na]⁺ | |
| | Calculated | 772.5210 |
| | Found | 772.5214. |

### EXAMPLE 20

Synthesis of Bis-De(3′-N-Methyl)-3′, 3′-N, N-Dihexyl-8, 9-Anhydro-Pseudoerythromycin A 6, 9-Hemiketal (EM739)

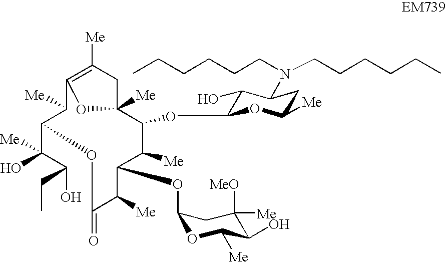

N,N-Diisopropylethylamine (116.0 μL, 0.666 mmol) and 1-bromohexane (93.6 μL, 0.666 mmol) were added in this order to acetinitrile (1.1 mL) solution of EM721 (22.9 mg, 0.0333 mmol) and stirred at 60° C. for 72 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM739 (20.1 mg, Yield: 71%, white powder).

| | |
|---|---|
| EM739: | m. p.: 158-160° C. |
| IR (KBr) ν: | 3490.0, 2958.3, 2931.3, 2871.5, 2858.0, |
| | 1702.8, 1459.8, 1376.9, 1319.1, 1265.1, |
| | 1166.7, 1126.2, 1083.8, 1016.3 cm⁻¹. |
| HRMS (FAB)m/z: | C₄₇H₈₆NO₁₂ [M + H]⁺ |

| | |
|---|---|
| Calculated | 856.6149 |
| Found | 856.6132. |

### EXAMPLE 21

Synthesis of Bis-De(3′-N-Methyl)-3′-N-(2-Fluoroethyl)-8, 9-Anhydro-Pseudoerythromycin A 6, 9-hemiketal (EM740)

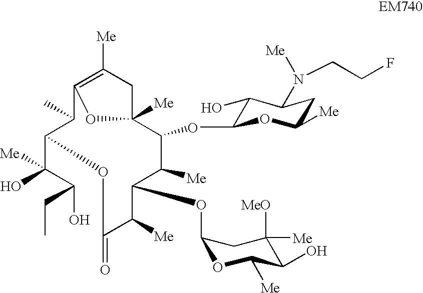

N,N-Diisopropylethylamine (347.7 μL, 1.996 mmol) and 1-bromo-2-fluoroethane (148.6 μL, 1.996 mmol) were added to dimethylformamide (3.3 mL) solution of EM703 (70.0 mg, 0.0998 mmol) at room temperature and stirred for 48 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM740 (36.0 mg, Yield: 48%, white powder). The raw material EM703 was recovered 25.5 mg (Yield: 36%).

| | |
|---|---|
| EM740: | m. p.: 138-140° C. |
| IR (KBr) ν: | 3480.8, 2973.7, 2937.1, 2879.2, 1704.8, |
| | 1457.9, 1376.9, 1319.1, 1265.1, 1166.7, |
| | 1126.2, 1114.7, 1076.1, 1049.1, 1035.6, |
| | 1016.3 cm⁻¹. |
| HRMS (FAB)m/z: | C₃₈H₆₆NO₁₂Fna [M + Na]⁺ |

| | |
|---|---|
| Calculated | 770.4467 |
| Found | 770.4469. |

### EXAMPLE 22

Synthesis of De(3′-N-Methyl)-3′-Cyanomethyl-8, 9-Anhydro-Pseudoerythromycin A 6, 9-Hemiketal (EM742)

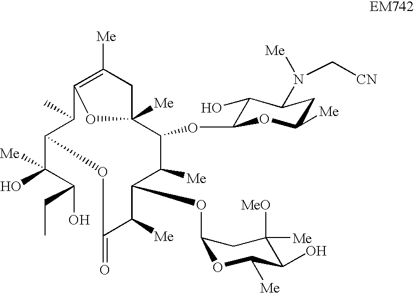

N,N-Diisopropylethylamine (320.9 μL, 1.847 mmol) and bromo acetinitrile (128.3 μL, 1.847 mmol) were added to dimethylformamide (3.1 mL) solution of EM703 (64.6 mg, 0.0921 mmol) at room temperature and stirred for 4 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM742 (53.1 mg, Yield: 78%, white powder).

| EM742: | m. p.: 110-112° C. | |
|---|---|---|
| IR (KBr) ν: | 3485.5, 2973.7, 2935.1, 2863.8, 1702.8, | |
| | 1456.0, 1382.7, 1319.1, 1265.1, 1166.7, | |
| | 1126.2, 1074.2, 1037.5, 1016.3 cm⁻¹. | |
| HRMS (FAB)m/z: | C₃₈H₆₄N₂O₁₂Na [M + Na]⁺ | |
| | Calculated | 763.4356 |
| | Found | 763.4377. |

### REFERENTIAL EXAMPLE 2

Synthesis of De(12-Hydroxy)-De[12-(1-Hydroxypropyl)]-12-Oxo-8, 9-Anhydro-Pseudoerythromycin A 6, 9-Hemiketal (EM705)

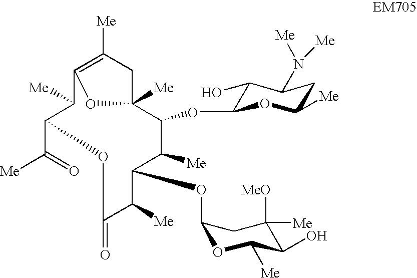

Lead tetraacetate (508.0 mg, 1.136 mmol) was added to dichloromethane (24.0 ml) solution of EM701 (508.0 mg, 0.701 mmol) and stirred at room temperature for 40 minutes. After confirming completion of the reaction by TLC, the reaction mixture was diluted with saturated brine-aqueous saturated sodium hydrogen carbonate (1:1, v/v) and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01) to obtain EM705 (282.7 mg, Yield: 61%, white powder).

| EM705: | m. p.: 108-112° C. | |
|---|---|---|
| IR (KBr) ν: | 3488, 2972, 2883, 1740, 1724, 1458, 1379, | |
| | 1244, 1165, 1107, 1093, 1076, 1055, 1034, | |
| | 1016 cm⁻¹. | |
| HRMS (FAB): | C₃₄H₅₈NO₁₁ [M + H]⁺ | |
| | Calculated | 656.4010 |
| | Found | 656.4021. |

### EXAMPLE 23

Synthesis of De(12-Hydroxy)-De[12-(1-Hydroxypropyl)]-12-Hydroxyoxime-8, 9-Anhydro-Pseudoerythromycin A 6, 9-Hemiketal (EM743) and the Salt Thereof

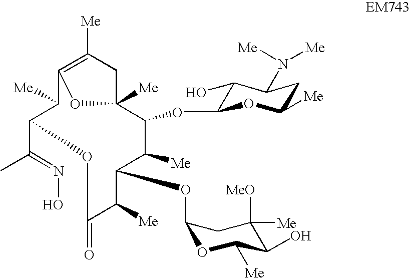

Pyridine (0.9 mL) was slowly added at 0° C. to ethanol (0.9 mL) solution of EM705 (116.5 mg, 0.1781 mmol) and hydroxylamine hydrochloride (32.0 mg, 0.533 mmol) and stirred for 3 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01→10:1:0.05 ) to obtain EM743 (114.5 mg, Yield: 96%, white powder).

| EM743: | m. p.: 141-143° C. | |
|---|---|---|
| IR (KBr) ν: | 3485.8, 2971.8, 2937.1, 2883.1, 1737.5, | |
| | 1459.8, 1378.9, 1255.4, 1247.7, 1166.7, | |
| | 1112.7, 1089.6, 1076.1, 1037.5, 1014.4 cm⁻¹. | |
| HRMS (FAB)m/z: | C₃₄H₅₉N₂O₁₁ [M + H]⁺ | |
| | Calculated | 671.4112 |
| | Found | 671.4108. |

### EXAMPLE 24

Synthesis of De[(3′-N-Methyl)-[3′-N-(3-Hydroxy-1-Propyl)]-8, 9-Anhydro-Pseudoerythromycin A 6, 9-Hemiketal (EM744)

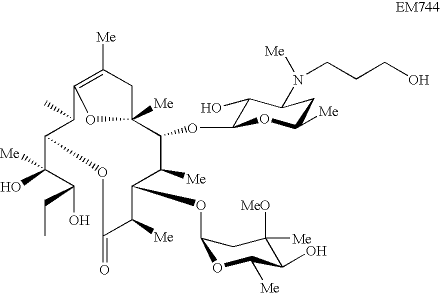

N,N-Diisopropylethylamine (338.3 μL, 1.942 mmol) and 3-bromo-1-propanol (175.6 μL, 1.942 mmol) were added to dimethylformamide (3.3 mL) solution of EM703 (68.1 mg, 0.0971 mmol) at room temperature and stirred for 48 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM744 (27.7 mg, Yield: 38%, white powder). The raw material EM703 was recovered 22.5 mg (Yield: 33%).

| EM744: | m. p.: 142-145° C. | |
|---|---|---|
| IR (KBr) ν: | 3478.8, 2973.7, 2937.1, 2877.3, 1700.9, | |
| | 1635.3, 1459.8, 1403.9, 1382.7, 1317.1, | |
| | 1267.0, 1166.7, 1126.2, 1114.7, 1076.1, | |
| | 1049.1, 1035.6, 1016.3 cm⁻¹. | |
| HRMS (FAB)m/z: | C₃₉H₆₉NO₁₃Na [M + Na]⁺ | |
| | Calculated | 782.4666 |
| | Found | 782.4667. |

### EXAMPLE 25

Synthesis of De(3′-N-Methyl)-3′-N-(2-Acetoxyethyl)-8, 9-Anhydro-Pseudoerythromycin A 6, 9-Hemiketal (EM745)

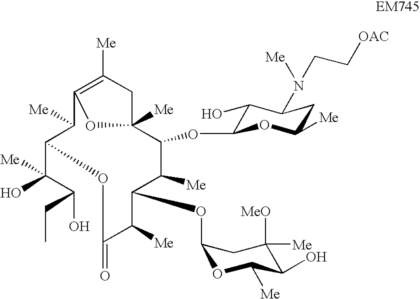

N,N-Diisopropylethylamine (106.8 μL, 0.613 mmol) and 2-bromoethylacetate (67.6 μL, 0.613 mmol) were added to dimethylformamide (1.0 mL) solution of EM703 (21.5 mg, 0.0307 mmol) at room temperature and stirred for 48 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM745 (6.0 mg, Yield: 25%, white powder).

| EM745: | m. p.: 131-133° C. | |
|---|---|---|
| IR (KBr) ν: | 3500.2, 3477.0, 2973.7, 2937.1, 2877.3, | |
| | 1735.6, 1700.9, 1457.9, 1376.9, 1319.1, | |
| | 1265.1, 1166.7, 1126.2, 1078.0, 1037.5, | |
| | 1016.3 cm⁻¹. | |
| HRMS (FAB)m/z: | C₄₀H₆₉NO₁₄Na [M + Na]⁺ | |
| | Calculated | 810.4615 |
| | Found | 810.4629 |

### EXAMPLE 26

Synthesis of De[12-(Hydroxypropyl)]-8, 9-Anhydro-Pseudo Erythromycin A 6, 9-Hemiketal (EM746)

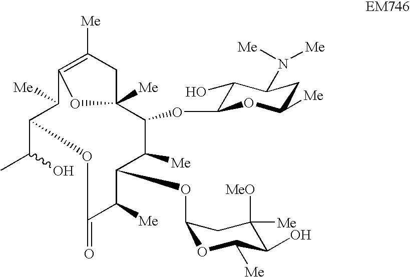

Sodium borohydride (21.8 mg, 0,575 mmol) was added to methanol (2.9 mL) solution of EM705 (37.7 mg, 0.0575 mmol) at −78° C. and stirred for 30 minutes. Temperature of the reaction mixture was increased to 0° C. and further stirred for 30 minutes. After confirming completion of the reaction by TLC, the reaction was terminated by adding acetone (0.5 ml). The reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM746 (35.8 mg, Yield: 95%, white powder)

| EM746: | m. p.: 116-118° C. | |
|---|---|---|
| IR (KBr) ν: | 3457.7, 2971.3, 2939.0, 1731.8, 1631.5, | |
| | 1457.9, 1378.9, 1265.1, 1166.7, 1110.8, | |
| | 1078.0, 1041.4, 1016.3 cm⁻¹. | |
| HRMS (FAB)m/z: | C₃₄H₅₉NO₁₁Na [M + Na]⁺ | |
| | Calculated | 680.3963 |
| | Found | 680.3963 |

### EXAMPLE 27

Synthesis of De(3′-Dimethylamino)-3′-Morpholino-8, 9-Anhydro-Pseudoerythromycin A 6, 9-Hemiketal (EM747)

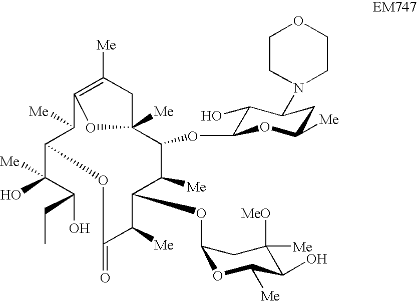

N,N-Diisopropylethylamine (45.8 μL, 0,263 mmol) and bis(2-bromoethyl) ether (33.1 μL, 0.263mmol) were added in this order to acetinitrile (2.6 mL) solution of EM721 (18.1 mg, 0.0263 mmol) and stirred at 80° C. for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM747 (12.0 mg, Yield: 60%, white powder).

| EM747: | m. p.: 139-142° C. | |
|---|---|---|
| IR (KBr) ν: | 3452.0, 2971.8, 2937.1, 2865.7, 1700.9, | |
| | 1646.9, 1457.9, 1380.8, 1319.1, 1265.1, | |
| | 1166.7, 1110.8, 1072.2, 1049.1, 1016.3 cm⁻¹. | |
| HRMS (FAB)m/z: | C₃₉H₆₇NO₁₃Na [M + Na]⁺ | |
| | Calculated | 780.4510 |
| | Found | 780.4529 |

### EXAMPLE 28

Synthesis of De(3′-Dimethylamino)-3′-[Hexahydro-1(1H)-Azepinyl]-8, 9-Anhydro-Pseudoerythromycin A 6, 9-Hemiketal (EM748)

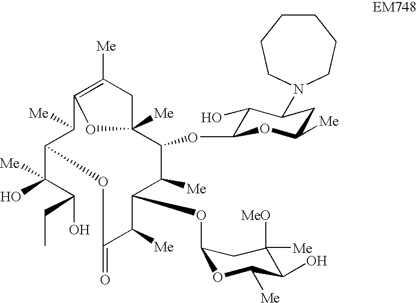

N,N-Diisopropylethylamine (49.5 μL, 0,284 mmol) and 1,6-dibromohexane (43.6 μL, 0.284 mmol) were added in this order to acetinitrile (2.8 ml) solution of EM721 (19.5 mg, 0.0284 mmol) and stirred at 80° C. for 24 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM748 (11.7 mg, Yield: 54%, white powder).

| EM748: | m. p.: 120-123° C. | |
|---|---|---|
| IR (KBr) ν: | 3430.7, 2971.8, 2933.2, 2858.0, 1708.6, | |
| | 1629.6, 1457.9, 1378.9, 1319.1, 1263.1, | |
| | 1166.7, 1112.7, 1083.8, 1047.2, 1016.3 cm⁻¹. | |
| HRMS (FAB)m/z: | C₄₁H₇₂NO₁₂ [M + H]⁺ | |
| | Calculated | 770.5054 |
| | Found | 770.5062. |

### EXAMPLE 29

Synthesis of Bis-De(3′-N-Methyl)-3′, 3′-N, N-Di-(10-Bromo-1-Decanyl)-8, 9-Anhydro-Pseudoerythromycin A 6, 9-Hemiketal (EM749)

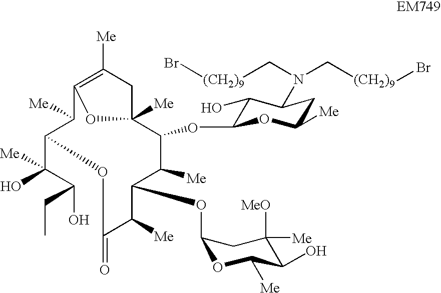

N,N-Diisopropylethylamine (45.6 μL, 0,262 mmol) and 1,10-dibromodecane (58.9 μL, 0.262 mmol) were added in this order to acetinitrile (2.6 mL) solution of EM721 (18.0 mg, 0.0262 mmol) and refluxed at 80° C. for 36 hours. After confirming completion of the reaction by TLC, the reaction mixture was diluted with water and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=20:1:0.1) to obtain EM749 (14.9 mg, Yield: 51%, white powder).

| EM749: | m. p.: 132-134° C. | |
|---|---|---|
| IR (KBr) ν: | 3448.1, 2929.3, 1700.9, 1629.6, 1459.8, | |
| | 1375.0, 1319.1, 1267.0, 1166.7, 1126.2, | |
| | 1081.9, 1049.1, 1016.3 cm⁻¹. | |
| HRMS (FAB)m/z: | C₅₅H₁₀₀NO₁₂Br₂ [M + H]⁺ | |
| | Calculated | 1126 |
| | Found | 1126. |

### EXAMPLE 30

Synthesis of De(12-Hydroxy)-De[12-(Hydroxypropyl)]-12-Amino-8,9-Anhydro-Pseudoerythromycin A 6, 9-Hemiketal (EM750)

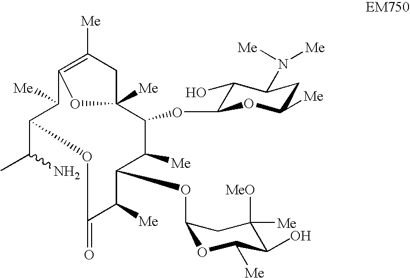

Molybdenum oxide (IV) (10.0 mg, 0,0694 mmol) and sodium borohydride (10.5 mg, 0.277 mmol) were added to ethanol (2.3 mL) solution of EM743 (15.5 mg, 0.0231 mmol) at 0° C. and stirred for 4 hours. After confirming completion of the reaction by TLC, the reaction was terminated by adding acetone (0.5 mL), and the reaction mixture was diluted with saturated brine-aqueous saturated sodium hydrogen carbonate (1:1, v/v) and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:1:0.1) to obtain EM750 (13.4 mg, Yield: 88%, white powder).

| EM750: | m. p.: 104-107° C. | |
|---|---|---|
| IR (KBr) ν: | 3448.1, 2971.8, 2935.1, 1729.8, 1629.6, | |
| | 1457.9, 1378.9, 1259.3, 1166.7, 1114.7, | |
| | 1078.0, 1039.4, 1016.3 cm⁻¹. | |
| HRMS (FAB)m/z: | C₃₄H₆₀N₂O₁₀Na [M + Na]⁺ | |
| | Calculated | 679.4145 |
| | Found | 679.4117. |

### REFERENTIAL EXAMPLE 3

Synthesis of De (3′-N-Methyl)-De (12-Hydroxy)-De-[12-(1-Hydroxy Propyl)]-12-Oxo-8, 9-Anhydro-Pseudoerythromycin A 6, 9-Hemiketal (EM706)

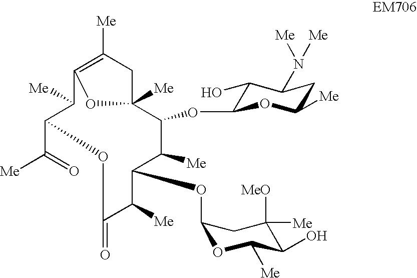

Lead tetraacetate (508.0 mg, 1.136 mmol) was added to dichloromethane (24.0 ml) solution of EM701 (508.0 mg, 0.701 mmol) and stirred at room temperature for 40 minutes. After confirming completion of the reaction by TLC, the reaction mixture was diluted with saturated brine-aqueous saturated sodium hydrogen carbonate (1:1, v/v) and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=10:0.5:0.01) to obtain EM706 (71.6 mg, Yield: 16%, white powder).

| EM706: | m. p.: 176-179° C. | |
|---|---|---|
| IR (KBr) ν: | 3468, 2966, 2852, 2360, 1736, 1718, 1558, | |
| | 1462, 1379, 1246, 1165, 1126, 1099, 1076, | |
| | 1038, 1016 cm⁻¹. | |
| HRMS (FAB)m/z: | C₃₃H₅₆NO₁₁ [M + H]⁺ | |
| | Calculated | 642.3853 |
| | Found | 642.3866. |

### EXAMPLE 31

Synthesis of De(3′-N-Methyl)-De[12-(1-Hydroxypropyl)]-8, 9-Anhydro-Pseudoerythromycin A 6, 9-Hemiketal (EM751)

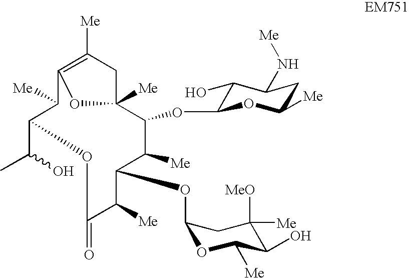

Sodium borohydride (22.9 mg, 0.605 mmol) was added to methanol (3.0 mL) solution of EM706 (38.8 mg, 0.0605 mmol) at 0° C. and stirred for 1 hour. After confirming completion of the reaction by TLC, the reaction was terminated by adding acetone (0.5 mL), and the reaction mixture was diluted with saturated brine-aqueous saturated sodium hydrogen carbonate (1:1, v/v) and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM751 (31.4 mg, Yield: 81%, white powder).

| | |
|---|---|
| EM751: | m. p.: 123-125° C. |
| IR (KBr) ν: | 3504.0, 2448.1, 2971.8, 2935.1, 1729.8, |
| | 1664.3, 1594.8, 1457.9, 1378.9, 1334.1, |
| | 1265.1, 1166.7, 1126.2, 1078.0, 1041.4, |
| | 1016 cm⁻¹. |
| HRMS (FAB)m/z: | C₃₃H₅₈NO₁₁ [M + H]⁺ |

| | |
|---|---|
| Calculated | 644.3987 |
| Found | 644.4011 |

### EXAMPLE 32

Synthesis of De(3-O-Cladinosyl)-De(3′-N-Methyl)-8, 9-Anhydrous-Pseudoerythromycin A 6, 9-Hemiketal (EM754)

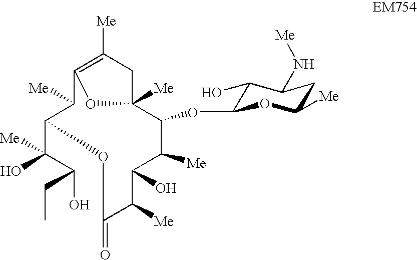

p-toluenesulfonic acid monohydrate (53.9 mg, 0.283 mmol) was added to dimethylformamide (3.8 mL) solution of EM703 (132.4 mg, 0.189 mmol) and stirred at 50° C. for 6 hours. After confirming completion of the reaction by TLC, the reaction mixture was diluted with water, adjusted to pH 8 by adding saturated aqueous sodium hydrogen carbonate and extracted with dichloromethane. The organic layer was dehydrated by adding sodium sulfate, filtered to remove the sodium sulfate, and removed the solvent to obtain crude substance. The crude substance was purified by silica gel column chromatography (chloroform:methanol:aqueous ammonia=15:1:0.1) to obtain EM754 (50.2 mg, Yield: 49%, white powder).

| EM754: | m. p.: 218-221° C. | |
|---|---|---|
| IR (KBr) ν: | 3432.7, 2969.8, 2927.4, 2858.0, 1708.6, | |
| | 1629.6, 1457.9, 1405.9, 1380.8, 1319.1, | |
| | 1270.9, 1232.3, 1130.1, 1078.0, 1039.4 cm⁻¹. | |
| HRMS (FAB)m/z: | C₂₈H₄₉NO₉Na [M + Na]⁺ | |
| | Calculated | 566.3305 |
| | Found | 566.3311. |

### EFFECT OF THE INVENTION

Novel pseudoerythromycin of the present invention has decreased antibacterial activity and increased antiinflammatory action, and is expected as the novel antiinflammatory agent.

1. A pseudoerythromycin derivative represented by the formula [I], 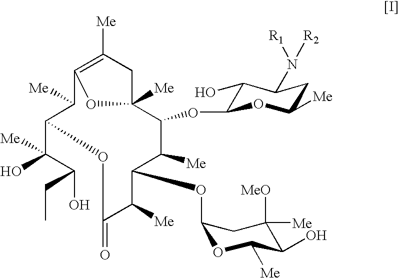
wherein R₁ and R₂ are same or different and each represents H, alkyl, alkynyl, acyl, or sulfonyl, in which these groups may optionally have substituents, and Me indicates methyl, wherein R₁ is Me or I—Pr, R₂ is not H.

2. A compound according to claim 1 which is bis-de(3′-N-methyl)-8, 9-anhydro-pseudoerythromycin A 6, 9-hemiketal or salt thereof.

3. A compound according to claim 1 which is bis-de(3′-N-methyl)-3′-N-ethyl-8, 9-anhydro-pseudoerythromycin A 6, 9-hemiketal or salt thereof.

4. A compound according to claim 1 which is bis-de(3′-N-methyl)-3′, 3′-N, N-diethyl-8, 9-anhydro-pseudoerythromycin A 6, 9-hemiketal or salt thereof.

5. A compound according to claim 1 which is bis-de(3′-N-methyl)-3′-N-propyl-8, 9-anhydro-pseudoerythromycin A 6, 9-hemiketal or salt thereof.

6. A compound according to claim 1 which is bis-de(3′-N-methyl)-3′, 3′-N, N-dipropyl-8, 9-anhydro-pseudoerythromycin A 6, 9-hemiketal or salt thereof.

7. A compound according to claim 1 which is bis-de(3′-N-methyl)-3′-N-(2-propyl)-8, 9-anhydro-pseudoerythromycin A 6, 9-hemiketal or salt thereof.

8. The derivative according to claim 1 wherein the compound represented by the general formula [I] has promoting action for differentiation-induction from monocyte to macrophage.

9. The derivative according to claim 1 wherein the compound represented by the general formula [I] has a suppressive effect against bleomycin-induced pulmonary fibrosis.

10. The derivative according to claim 1 wherein the compound represented by the general formula [I] has suppressive effect against pneumonia caused by influenza viral infection.

11. A pseudoerythromycin derivative represented by the formula [III], 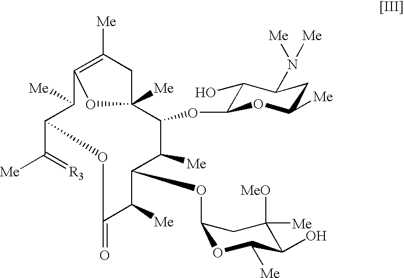
wherein R₃ is O or NOH, and Me indicates methyl.

12. The derivative according to claim 11 wherein the compound represented by the general formula [III] has promoting action for differentiation-induction from monocyte to macrophage.

13. The derivative according to claim 11 wherein the compound represented by the general formula [III] has a suppressive effect against bleomycin-induced pulmonary fibrosis.

14. The derivative according to claim 11 wherein the compound represented by the general formula [III] has suppressive effect against pneumonia caused by influenza viral infection.

15. A pseudoerythromycin derivative represented by the formula [IV], 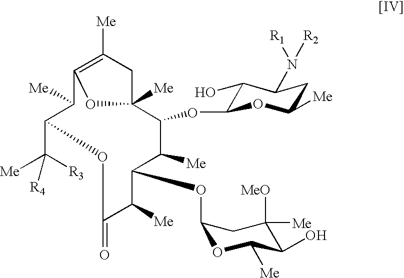
wherein R₁ and R₂ are same or different and each represents H or methyl, R₃ and R₄ represent H, hydroxyl or amino, and Me indicates methyl.

16. The derivative according to claim 15 wherein the compound represented by the general formula [IV] has promoting action for differentiation-induction from monocyte to macrophage.

17. The derivative according to claim 15 wherein the compound represented by the general formula [IV] has a suppressive effect against bleomycin-induced pulmonary fibrosis.

18. The derivative according to claim 15 wherein the compound represented by the general formula [IV] has suppressive effect against pneumonia caused by influenza viral infection.

19. A pseudoerythromycin derivative represented by the formula [V], 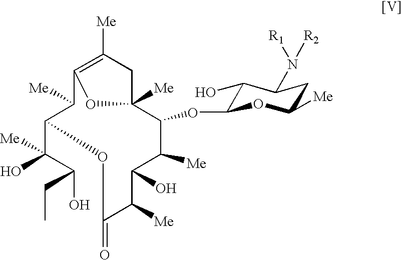
wherein R₁ and R₂ are same or different and each represents H or methyl, and Me indicates methyl.

20. The derivative according to claim 19 wherein the compound represented by the general formula [V] has promoting action for differentiation-induction from monocyte to macrophage.

21. The derivative according to claim 19 wherein the compound represented by the general formula [V] has a suppressive effect against bleomycin-induced pulmonary fibrosis.

22. The derivative according to claim 19 wherein the compound represented by the general formula [V] has suppressive effect against pneumonia caused by influenza viral infection.
